(19) United States
(12) Patent Application Publication
(10) Pub. No.: US 2025/0109089 A1
(43) Pub. Date: Apr. 3, 2025

(54) PROCESS FOR PRODUCING DIESTERS, AND DIESTERS OBTAINED THEREBY

(71) Applicant: TOTALENERGIES ONETECH, Courbevoie (FR)

(72) Inventors: Delphine CROZET, Solaize (FR); Alice LIMOGES, Solaize (FR)

(73) Assignee: TOTALENERGIES ONETECH, Courbevoie (FR)

(21) Appl. No.: 18/719,971

(22) PCT Filed: Dec. 15, 2022
(86) PCT No.: PCT/EP2022/086078

§ 371 Date: Jun. 14, 2024

(30) Foreign Application Priority Data
Dec. 16, 2021 (FR) FR2113737

(51) Int. Cl. C07C 67/08; C10M 105/36

(52) U.S. Cl. CPC C07C 67/08; C10M 105/36

(57) ABSTRACT

The invention relates to a method for preparing at least one diester, said method comprising:
a) a transformation reaction of the acid function of at least one hydroxycarboxylic acid, or of the ester function of at least one ester of hydroxycarboxylic acid and alcohol, to an alcohol function to obtain at least one diol, said hydroxycarboxylic acid having 11 to 25 carbon atoms;
b) an esterification reaction of the diol by at least one saturated or unsaturated acid having 2 to 24 carbon atoms, to form at least one diester.

The invention also relates to the use of the diesters as base oil in a lubricant composition, or as emollient in a cosmetic or pharmaceutical composition.

### TECHNICAL FIELD OF THE INVENTION

The invention relates to a method of preparing diesters with a method that is simple to implement starting from widely accessible raw materials.

The invention also relates to diesters obtainable by the method of the invention and to the use thereof as base oil in a lubricant composition. The diester composition of the invention can also be used in cosmetic or pharmaceutical compositions. The invention also concerns a cosmetic or pharmaceutical composition.

### PRIOR ART

Lubricant compositions, also called lubricants, are widely used to reduce friction between the surfaces of moving parts, thereby reducing wear and preventing degradation on the surface of these parts. Lubricants typically comprise a base oil and one or more functional additives.

When lubricant compositions are subjected to high stresses (i.e. strong pressures) when in service, those lubricant compositions having a base oil composed of hydrocarbons tend to degrade, giving rise to damage to parts.

Lubricant manufacturers must constantly improve their formulations to meet increasing requirements in terms of fuel economy whilst maintaining engine cleanliness and reducing emissions. These requirements compel manufacturers to review their formulation capabilities and/or to research novel base oils able to meet performance requirements.

For the manufacture of lubricants such as engine oils, transmission fluids, gear oils, industrial lubricant oils, metal-working oils etc., the starting product is typically an oil of petroleum origin of lubricant grade produced by a refinery, or a suitable polymerized petrochemical fluid. To this base oil, additives are added to improve properties and performance such as an increase in lubricating power, anti-wear and anti-corrosion properties, resistance of the lubricant to heat and/or oxidation. Therefore, various additives such as antioxidants, corrosion inhibitors, dispersants, defoaming agents, metal deactivators and other additives able to be used in lubricant formulations, can be added in conventional effective amounts.

Environmental concerns and restrictions mean that industrialists are driven to find an alternative to sources of petroleum (fossil) sources. Oils of vegetable or animal origin have proved to be sources of interest for base oils. In particular, these oils of vegetable or animal origin can be converted to acids or esters via conventional processes.

In the API classification of base oils, esters are referenced as base oils in Group V. Synthetic esters can be used both as oil base and as lubricant additive. Compared with less costly mineral oils but less safe for the environment, synthetic esters have been especially used as base oils for cases in which viscosity/temperature behaviour must meet strict requirements. Ever more important issues of environmental acceptability and biodegradability have prompted the desire to find alternatives to mineral oils as raw material in lubricant applications.

The cosmetic, dermatological, and also pharmaceutical markets are all seeking ingredients of biological origin for the formulation of their products. Although biosourced active substances, emulsifiers and vegetable oils have undergone strong development in recent years and are now widely available on the market, emollients of 100% bio-based origin still remain few.

The emollients currently used in cosmetics are either isoparaffins derived from the petrochemical industry (chiefly isododecane and isohexadecane), white oils, silicone oils or ester-based oils (synthetic or natural). Isoparaffins, white oils and silicone oils are widely distributed since they are very stable and odourless, but they are not derived from a renewable source. Although volatile silicones such as cyclomethicone have long been considered to be emollients and solvents harmless for the skin (International Journal of Toxicology, Vol. 10, N^{o} I, pp. 9-19, 1991), some fears have been expressed in recent years regarding the potential deleterious effects thereof on the environment, even on human health (the case in particular for octamethylcyclotetrasiloxane).

Environmental concerns and restrictions have led industrialists to finding alternatives to sources of petroleum (fossil) origin. Oils of vegetable or animal origin have therefore proved to be sources of interest for base oils or emollients. In particular, these oils of vegetable or animal origin can be converted to acids or esters with conventional processes. These acids can then be converted to unsaturated alcohols for example from a triglyceride oil, with one or more hydrogenation steps of the fatty acids or methyl esters.

Document US 2010/120642 discloses diester-based lubricant compositions obtained via epoxide intermediates. Document U.S. Pat. No. 5,008,126 discloses diol diesters in which the ester functions are separated by one or two methylene groups. These diol diesters are used as substitute for fats in foodstuffs.

Document WO2008100822 discloses diester-based lubricant compositions obtained via an epoxide intermediate. The diesters described in this document exhibit a low viscosity index.

Document WO2011005604 discloses lubricant compositions comprising a mixture of isomers of at least two esters, obtained by epoxidation of a double carbon-carbon bond, followed by opening of the epoxide on each of the two positions. The diester composition described in this document comprises a mixture of diesters differing though their central structure (structure between the two ester functions).

The invention sets out to provide a diester composition having strong selectivity for a specifically targeted diester, with high conversion rate. In addition, this diester composition can be obtained from raw materials of vegetable or animal origin.

### SUMMARY OF THE INVENTION

The invention relates to a method of preparing at least one diester, said method comprising:
a) a transformation reaction of the acid function of at least one hydroxycarboxylic acid, or of the ester function of at least one ester of hydroxycarboxylic acid and alcohol, to an alcohol function to obtain at least one diol, said hydroxycarboxylic acid having 11 to 25 carbon atoms;
b) an esterification reaction of the diol via at least one saturated or unsaturated acid having 2 to 24 carbon atoms, to form at least one diester.

In one embodiment, the method of the invention, after step a) and before step b), also comprises a step a-bis) to purify the composition of diols obtained after step a) to increase the diol content of the diol composition obtained after step a), said purification step a-bis) preferably comprising at least one crystallization step of the diol.

In one embodiment, the method of the invention, before step a), also comprises a preliminary purification step of the composition of hydroxycarboxylic acid or hydroxycarboxylic acid ester to increase the content of hydroxycarboxylic acid or hydroxycarboxylic acid ester in the composition of hydroxycarboxylic acid or hydroxycarboxylic acid ester used at following step a), said preliminary purification step preferably comprising at least one crystallization step of the hydroxycarboxylic acid or hydroxycarboxylic acid ester.

Preferably, the hydroxycarboxylic acid meets formula (1) and/or the saturated or unsaturated acid meets formula (2):

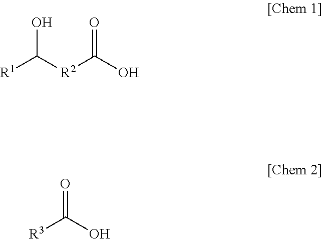

where:
R¹ is a linear or branched alkyl or alkenyl radical having 1 to 22 carbon atoms, preferably 3 to 18 carbon atoms, more preferably 5 to 12 carbon atoms,
R² is a divalent linear or branched alkyl or alkenyl radical having 1 to 22 carbon atoms, preferably 4 to 22 carbon atoms, more preferably 8 to 18 carbon atoms, with the proviso that that the total number of carbon atoms of R¹ and R² ranges from 9 to 23, preferably 9 to 20 carbon atoms, more preferably 10 to 18 carbon atoms.
R³ is a monovalent linear or branched alkyl radical having 1 to 19 carbon atoms, preferably a linear or branched alkyl having to 2 to 17 carbon atoms, advantageously a linear alkyl having 4 to 12 carbon atoms.

Preferably, the diol meets formula (3) and the diester meets formula (4):

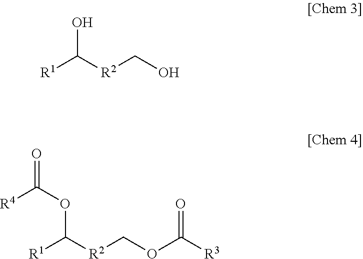

where:
R¹ is a linear or branched alkyl radical having 1 to 22 carbon atoms, or a linear or branched alkenyl radical having 2 to 22 carbon atoms, preferably an alkyl or alkenyl radical having 3 to 18 carbon atoms, more preferably 5 to 12 carbon atoms;
R² is a divalent linear or branched alkyl radical having 1 to 22 carbon atoms, or a linear or branched alkenyl radical having 2 to 22 carbon atoms, preferably or alkyl or alkenyl radical having 4 to 22 carbon atoms, more preferably 8 to 18 carbon atoms, with the proviso that the total number of carbon atoms of R¹ and R² ranges from 9 to 23, preferably 9 to 20 carbon atoms, more preferably 10 to 18 carbon atoms;
R³ and R⁴ are each independently a monovalent linear or branched alkyl radical having 1 to 19 carbon atoms, or a monovalent linear or branched alkenyl radical having 1 to 18 carbon atoms, preferably a linear or branched alkyl or alkenyl having 2 to 17 carbon atoms, advantageously a linear alkyl or alkenyl having 2 to 12 carbon atoms.

The invention also concerns a composition of diester(s) comprising at least one diester of formula (4):

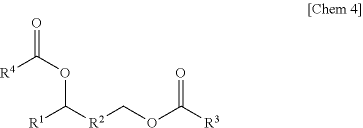

where:
R¹ is a linear or branched alkyl radical having 1 to 22 carbon atoms, or a linear or branched alkenyl radical having 2 to 22 carbon atoms, preferably an alkyl or alkenyl radical having 3 to 18 carbon atoms, more preferably 5 to 12 carbon atoms;
R² is a divalent linear or branched alkyl radical having 1 to 22 carbon atoms, or a linear or branched alkenyl radical having 2 to 22 carbon atoms, preferably an alkyl or alkenyl radical having 4 to 22 carbon atoms, more preferably 8 to 18 carbon atoms,
with the proviso that the total number of carbon atoms of R¹ and R² ranges from 9 to 23, preferably 9 to 20 carbon atoms, more preferably 10 to 18 carbon atoms;
R³ and R⁴ are each independently a monovalent linear or branched alkyl radical having 1 to 19 carbon atoms, or a monovalent linear or branched alkenyl radical having 1 to 19 carbon atoms, or a monovalent linear or branched alkenyl radical having 1 to 18 carbon atoms, preferably a linear or branched alkyl or alkenyl having 2 to 17 carbon atoms, advantageously a linear alkyl or alkenyl having 2 to 12 carbon atoms,
said composition of diester(s) relative to the total weight of the composition of diester(s) comprising at least 70% by weight of diesters of formula (4) which only differ from each other by the groups R3 and R4.

In one embodiment, the composition of diester(s) of the invention is obtainable by the method of the invention.

In one embodiment, R³ and R⁴ are each independently a monovalent linear or branched alkyl radical having 1 to 19 carbon atoms, or a monovalent linear or branched alkenyl radical having 12 to 17 carbon atoms.

Preferably, the composition of diester(s) of the invention, relative to the total weight of the composition, comprises at least 70% by weight of a single diester meeting formula (4), preferably at least 80% by weight of a single diester meeting formula (4), preferably at least 90% by weight of a single diester meeting formula (4).

The invention also concerns the use of the composition of diester(s) of the invention as base oil in a lubricant composition or as emollient in a cosmetic or pharmaceutical composition.

Finally, the invention concerns a lubricant composition comprising the composition of diester(s) of the invention, and:
(i) at least one base oil differing from the diesters, and/or
(ii) at least one additive differing from the diesters.

Preferably, R³ and R⁴ are each independently a monovalent linear or branched alkyl radical having 1 to 19 carbon atoms, preferably a linear or branched alkyl having 2 to 17 carbon atoms, advantageously a linear alkyl having 2 to 12 carbon atoms.

The invention also concerns a cosmetic or pharmaceutical composition comprising (i) the composition of diester(s) of the invention and (ii) at least one fat and/or (iii) at least one cosmetic additive. Preferably, the composition of diester(s) comprises at least one diester of formula (4) where R³ and R⁴ are each independently a monovalent linear or branched alkenyl radical having 1 to 18 carbon atoms, preferably 2 to 17 carbon atoms, more preferably 12 to 17 carbon atoms.

With the method of the invention, it is possible to obtain good selectivity for the formation of a specific diester, without the forming of position isomer(s).

The method is simple to implement from available raw materials.

The method of the invention allows the obtaining of a diester composition having an improved viscosity index, in particular compared with diesters obtained by opening of an epoxy function.

### DETAILED DESCRIPTION OF THE INVENTION

The invention concerns a method of preparing a composition of diester(s), said method comprising:
a) a transformation reaction of the acid function of at least one hydroxycarboxylic acid, or of the ester function of at least one ester of hydroxycarboxylic acid and alcohol, to an alcohol function to obtain at least one diol, said hydroxycarboxylic acid having 11 to 25 carbon atoms;
b) an esterification reaction of the diol by at least one acid having 2 to 24 carbon atoms to form at least one diester.

In one embodiment, the method of the invention, before step a), comprises a preliminary step to purify a composition of hydroxycarboxylic acid or hydroxycarboxylic acid ester to increase the content of hydroxycarboxylic acid or hydroxycarboxylic acid ester in the composition of hydroxycarboxylic acid or hydroxycarboxylic acid ester. A purified composition of hydroxycarboxylic acid or hydroxycarboxylic acid ester is then obtained. In this embodiment, step a) of the method of the invention (reduction reaction) is implemented starting from the composition of hydroxycarboxylic acid or hydroxycarboxylic acid ester purified at the preliminary step.

Preferably, this preliminary purification step is performed by crystallization.

A composition of hydroxycarboxylic acid or hydroxycarboxylic acid ester may comprise impurities such as saturated or unsaturated acids (without hydroxyl function) or saturated or unsaturated esters (without hydroxyl function). Preliminary purification allows removal of these impurities to increase the content of hydroxycarboxylic acid or hydroxycarboxylic acid ester.

Preferably, the purified composition of hydroxycarboxylic acid or hydroxycarboxylic acid ester comprises at least 90% by weight of hydroxycarboxylic acid or hydroxycarboxylic acid ester, preferably at least 95% by weight of hydroxycarboxylic acid or hydroxycarboxylic acid ester, more preferably at least 98% by weight of hydroxycarboxylic acid or hydroxycarboxylic acid ester relative to the total weight of the composition of hydroxycarboxylic acid or of hydroxycarboxylic acid ester, relative to the total weight of the purified composition of hydroxycarboxylic acid or hydroxycarboxylic acid ester.

Typically, the purified composition of hydroxycarboxylic acid or hydroxycarboxylic acid ester comprises at most 10% by weight of impurities, preferably at most 5% by weight of impurities, relative to the total weight of the composition of hydroxycarboxylic acid or hydroxycarboxylic acid ester, said impurities comprising for example saturated or unsaturated acids (without hydroxyl function) and/or saturated or unsaturated esters (without hydroxyl function).

In one embodiment, the method of the invention, after step a) and before step b), comprises a step a-bis) to purify the composition of diols obtained after step a) to increase the content of diols in the composition of diols obtained after step a). If implementation thereof is necessary, this step a-bis) is preferably performed when the method does not comprise a preliminary purification step.

A composition of hydroxycarboxylic acid or hydroxycarboxylic acid ester may comprise impurities such as saturated or unsaturated acids (without hydroxyl function) or saturated or unsaturated esters (without hydroxyl function). The reduction step a) of the method of the invention may form monoalcohols from these impurities. This intermediate purification will allow the removal of initial impurities and of reaction by-products of these impurities (e.g. monoalcohols).

In one preferred embodiment, this purification step a-bis) is performed by crystallization.

Preferably, the purified composition of diols comprises at least 90% by weight of diols, preferably at least 95% by weight of diols, more preferably at least 98% by weight of diols, relative to the total weight of the purified diol composition.

In one embodiment, when a purification step is performed, the method of the invention comprises one or more treatment steps, in particular to remove the crystallization solvent is the purification step is a crystallization step. The treatment step may comprise a filtering step for example and a drying step.

The method of the invention may optionally comprise washing and/or additional purification steps. It is to be noted that the method of the invention may comprise one or more operations to separate the acid and/or hydroxycarboxylic acid, starting reagents of the method of the invention. For example, these operations can be stripping operations or distilling operations.

The method of the invention may also comprise one or more washing operations to separate the homogeneous catalyst from the product resulting from the method of the invention, or one or more filtering steps to separate the heterogeneous catalyst from the product resulting from the method of the invention.

As a preliminary, it is noted that in the following description and claims, the expression «between . . . and», «from . . . to» is to be construed as including the cited limits.

### Composition C1 of Hydroxycarboxylic Acids

The method of the invention, as reagent, uses at least one hydroxycarboxylic acid or at least one ester of hydroxycarboxylic acid and alcohol.

In the meaning of the present invention, the hydroxycarboxylic acid comprises at least one carboxylic acid function (—COOH) and at least one hydroxyl function (—OH). Preferably, the hydroxycarboxylic acid comprises a single carboxylic acid function and a single hydroxyl function.

The hydroxycarboxylic acid can be saturated or unsaturated. Preferably, the hydroxycarboxylic acid is a saturated acid which typically does not comprise a function other than the carboxylic acid function and hydroxyl function.

If it is unsaturated, the hydroxycarboxylic acid is preferably monounsaturated.

In one preferred embodiment, the hydroxycarboxylic acid comprises a linear or branched alkyl chain, preferably linear.

The hydroxycarboxylic acid able to be used in the invention has 11 to 25 carbon atoms, preferably 12 to 24 carbon atoms, more preferably 14 to 20 carbon atoms.

In one preferred embodiment, the hydroxycarboxylic acid used in the invention comprises at least one hydroxyl function carried by a secondary carbon atom (compound of so-called secondary alcohol type).

In one embodiment, the hydroxycarboxylic acid used in the invention comprises at least one carboxylic acid function carried by a primary carbon atom.

Preferably the hydroxycarboxylic acid meets formula (1):

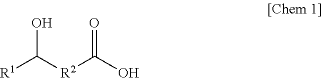

where:
R¹ is a linear or branched alkyl or alkenyl radical having 1 to 22 carbon atoms, preferably 3 to 18 carbon atoms, more preferably 5 to 12 carbon atoms,
R² is a divalent linear or branched alkyl or alkenyl radical having 1 to 22 carbon atoms, preferably 4 to 22 carbon atoms, more preferably 8 to 18 carbon atoms,
with the proviso that the total number of carbon atoms of R¹ and R² ranges from 9 to 23, preferably from 9 to 20 carbon atoms, more preferably 10 to 18 carbon atoms.

Preferably, in formula (1), if the hydroxycarboxylic acid is unsaturated, then only one of the radicals among R¹ and R² is monounsaturated, the other radical being saturated.

Preferably, in formula (1):
R¹ is a linear or branched alkyl radical having 1 to 22 carbon atoms, preferably 3 to 18 carbon atoms, more preferably 5 to 12 carbon atoms,
R² is a divalent linear or branched alkyl radical having 1 to 22 carbon atoms, preferably 4 to 22 carbon atoms, more preferably 8 to 18 carbon atoms,
with the proviso that the total number of carbon atoms of R¹ and R² ranges from 9 to 20 carbon atoms, preferably 10 to 18 carbon atoms.

Preferably, in formula (1):
R¹ is a linear alkyl radical having 3 to 18 carbon atoms, preferably 5 to 12 carbon atoms,
R² is a divalent linear alkylene radical having 4 to 22 carbon atoms, preferably 8 to 18 carbon atoms,
with the proviso that the total number of carbon atoms of R¹ and R² ranges from 9 to 20 carbon atoms, preferably 10 to 18 carbon atoms.

In one embodiment, the conversion reaction to obtain at least one diol is performed using as reagent an ester of hydroxycarboxylic acid (such as defined in the invention) and alcohol. Reduction at one same step will allow reducing of the ester function to an alcohol function.

In the meaning of the present invention, the expression «ester derived from hydroxycarboxylic acid» or the expression «hydroxycarboxylic acid ester» or the expression «ester of hydroxycarboxylic acid and alcohol» designates an ester obtained by reaction of a hydroxycarboxylic acid and alcohol.

The hydroxycarboxylic acid on which the ester of hydroxycarboxylic acid and alcohol is based, preferably has one or more of the characteristics or preferences described above.

The alcohol used for the hydroxycarboxylic acid ester is preferably a saturated monoalcohol preferably having 1 to 18 carbon atoms, preferably 1 to 16 carbon atoms, preferably 1 to 12 carbon atoms.

Preferably, the hydroxycarboxylic acid used in the invention is 12-hydroxystearic acid. If the ester form is used in the method of the invention, the ester in one particular embodiment could be the methyl or ethyl ester of 12-hydroxystearic acid.

In one embodiment, the hydroxycarboxylic acid is unsaturated and is ricinoleic acid.

In one embodiment, before the reduction step a), the method further comprises a preliminary purification step of the composition C1 of hydroxycarboxylic acid(s) or ester(s) thereof. Said preliminary purification step preferably comprises crystallization of the diol.

The crystallization step can be performed with a solvent or mixture of solvents in one or more crystallization steps. The solvent(s) can be chosen from among heptane, toluene, acetone, methanol, ethanol, ethyl acetate, a mixture thereof. The ratio between the volume of crystallization solvent (in mL) and weight of diol (in g) preferably ranges from 1 to 50, preferably 2 to 30.

The method of the invention, before step a) and before the optional preliminary purification step, may comprise a step to provide a composition C1 of hydroxycarboxylic acid(s) or ester(s) thereof.

Typically, the reduction reaction of the invention (conversion of acid or ester function to an alcohol function) is performed in the presence of a composition C1 comprising at least 50% by weight of hydroxycarboxylic acid(s) or ester(s) thereof, preferably at least 70% by weight, more preferably at least 75% by weight, even at least 80% by weight of hydroxycarboxylic acid(s) or ester(s) thereof, relative to the total weight of composition C1.

In one embodiment, composition C1 used in the invention comprises at least 50% by weight of 12-hydroxystearic acid or methyl or ethyl ester of 12-hydroxystearic acid, preferably at least 70% by weight, more preferably at least 80% by weight of 12-hydroxystearic acid or methyl or ethyl ester of 12-hydroxystearic acid, relative to the total weight of composition C1.

Preferably, composition C1 used in the invention comprises at least 50% by weight of 12-hydroxystearic acid, preferably at least 70% by weight, more preferably at least 80% by weight 12-hydroxystearic acid relative to the total weight of composition C1.

If the method comprises a preliminary purification step of composition C1, then the reduction step a) of the method is preferably performed in the presence of a composition C1′ comprising at least 90% by weight of hydroxycarboxylic acid or hydroxycarboxylic acid ester, preferably at least 95% by weight of hydroxycarboxylic acid or hydroxycarboxylic acid ester, more preferably at least 98% by weight of hydroxycarboxylic acid or hydroxycarboxylic acid ester, relative to the total weight of purified composition C1′ of hydroxycarboxylic acid or hydroxycarboxylic acid ester.

Preferably, composition C1′ able to be used in the invention comprises at least 90% by weight of 12-hydroxystearic acid, preferably at least 95% by weight, more preferably at least 98% by weight of 12-hydroxystearic acid, relative to the total weight of composition C1′.

Composition C1 or C1′ can be commercially available.

### Composition C2 of Saturated or Unsaturated Acids

The method of the invention for esterification step b) comprises at least one acid having 2 to 20 carbon atoms, preferably an acid having 2 to 18 carbon atoms, as reagent to react on the alcohol functions of the diol.

Preferably, said at least one acid is a monoacid. The acid can be a linear or branched, saturated or unsaturated acid.

In one embodiment, said at least one saturated acid meets formula (2):

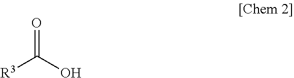

where R³ is a monovalent linear or branched alkyl radical having 1 to 19 carbon atoms, or a monovalent linear or branched alkenyl radical having 1 to 18 carbon atoms, preferably a linear or branched alkyl or alkenyl having 2 to 17 carbon atoms, advantageously a linear alkyl or alkenyl having 2 to 12 carbon atoms.

Preferably, if R³ is an alkenyl, said alkenyl is monounsaturated.

In one embodiment, the acid is a saturated fatty acid and has 2 to 12 carbon atoms. With this chain length, it is possible to further optimize the cold behaviour properties of the diester composition obtained with the method, which is particularly preferred in lubricant applications.

In another embodiment, the acid is an unsaturated acid and has 8 to 18 carbon atoms. Unsaturated acids can be particularly preferred in diesters intended for cosmetic applications.

In one embodiment, the acid used in the invention is chosen from one or more acids from among: acetic acid, propanoic acid, butanoic acid, pentanoic acid, hexanoic acid, heptanoic acid, octanoic acid, nonanoic acid, decanoic acid, undecanoic acid, lauric acid, and mixture thereof; these acids can be linear or branched.

At the time of esterification, the method of the invention can use a single acid or mixture of several acids. Preferably, the method of the invention uses a single acid.

Typically, the method of the invention for the esterification step uses either solely saturated acids (i.e. from a composition of saturated acids having at least 90 weight % of saturated acids), or solely unsaturated acids (i.e. from a composition of unsaturated acids having at least 85 weight % of unsaturated acids).

At the time of esterification, it is also possible to envisage the use of a mixture of at least two different acids. The proportions can be adjusted as a function of the desired properties for the diester composition.

The two esterification reactions (on the two alcohol functions of the diol) can be conducted simultaneously or in sequence. When at least two different acids are used, it is possible to adjust the operating conditions to choose the saturated or unsaturated acid which will first react on the most reactive alcohol function of the diol. Therefore, the saturated or unsaturated acids can be added simultaneously or in sequence as a function of the desired diester.

The method of the invention is selective since it allows the obtaining of diesters that are specifically chosen and have a specific structure.

In one embodiment, the esterification reaction is conducted with a composition C2 of saturated acid(s) typically comprising at least 90% by weight of saturated acid(s), preferably at least 95% by weight, even at least 98% by weight of saturated acid(s) relative to the total weight of composition C2.

In another embodiment, the esterification reaction is conducted with a composition C2 of unsaturated acid(s) typically comprising at least 85% by weight of unsaturated acid(s), preferably at least 90% by weight, even at least 95% by weight of unsaturated acid(s), relative to the total weight of composition C2.

Composition C2 can be commercially available and can be of natural or synthetic origin, preferably of natural origin.

### Implementation of the Method of the Invention

The method of the invention uses two chemical reactions:
reduction reaction of the acid or ester function to an alcohol function, and
esterification reaction.

The two chemical reactions used in the method of the invention are typically conducted in the presence of one or more catalysts. Nevertheless, it is possible to carry out the diol esterification step b) without a catalyst.

### Reduction Reaction

Typically, the acid function of hydroxycarboxylic acid or ester function of the hydroxycarboxylic acid ester is transformed into an alcohol function via a reduction reaction. The reduction reaction can be performed with a hydrogenation step, or by contacting the hydroxycarboxylic acid or ester thereof with a reducing agent. Reducing methods of an acid or ester function to an alcohol function are known to skilled persons.

The reducing agent can be chosen from among metal hydrides, preferably lithium hydrides or sodium hydrides. For example, the reducing agent is chosen from among lithium aluminium hydride and sodium borohydride.

Typically, when contacting the hydroxycarboxylic acid or ester thereof with the reducing agent, the temperature of the mixture ranges from −10° C. to +15° C., preferably from −5° C. to +10° C.

The reducing step of the acid function of hydroxycarboxylic acid or ester function of the ester derived from hydroxycarboxylic acid can be carried out under conditions known to skilled persons.

The hydrogenation step can be performed under the following conditions:
temperature ranging from 100 to 450° C., preferably from 200 to 400° C., more preferably from 250 to 380° C., and/or
pressure ranging from 30 to 300 bar, preferably from 50 to 250 bar, more preferably from 150 to 230 bar.

One nonlimiting example of hydrogenation catalyst allowing the reducing of acid or ester functions to an alcohol is copper chromite.

In one nonlimiting embodiment, hydrogenation is performed at a temperature of 350° C. and pressure of 200 bar in the presence of hydrogen and copper chromite.

In one embodiment, the diol formed after the reducing step meets formula (3):

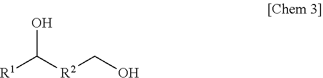

where:
R¹ is a linear or branched alkyl radical having 1 to 22 carbon atoms, or a linear or branched alkenyl radical having 2 to 22 carbon atoms, preferably an alkyl or alkenyl radical having 3 to 18 carbon atoms, more preferably 5 to 12 carbon atoms;
R² is a divalent linear or branched alkyl radical having 1 to 22 carbon atoms, or a linear or branched alkenyl radical having 2 to 22 carbon atoms, preferably an alkyl or alkenyl radical having 4 to 22 carbon atoms, more preferably 8 to 18 carbon atoms,
with the proviso that the total number of carbon atoms of R¹ and R² ranges from 9 to 23, preferably 9 to 20 carbon atoms, more preferably 10 to 18 carbon atoms.

If the hydroxycarboxylic acid or ester thereof is unsaturated, it is then possible to choose the reaction conditions such that the reduction step a) does not reduce the double carbon-carbon bonds of the hydroxycarboxylic acid or ester thereof. Mention can be made for example of copper chromite.

In one embodiment, the reduction reaction uses a hydroxycarboxylic acid of formula (1) to obtain a diol of formula (3), with the following scheme:

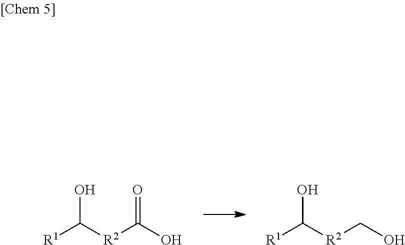

where R¹ and R² are such as defined in formulas (1) and (3) respectively.

At the reduction step of hydroxycarboxylic acid, by-products may form. Among these by-products monoalcohols can be cited obtained by reducing the acid function of acid impurities which may be contained in composition C1 of hydroxycarboxylic acid. For example, the composition of 12-hydroxystearic acid, in addition to 12-hydroxystearic acid, may comprise palmitic acid and/or stearic acid.

In one embodiment, after the reduction step a) and before the esterification step b), the method further comprises a step a-bis) to purify the composition of diol(s) obtained after step a). Said purification step a-bis) preferably comprises crystallization of the diol.

The crystallization step can be performed with a solvent or mixture of solvent in one or more crystallization steps. The solvent(s) can be chosen from among heptane, toluene, acetone, methanol, ethanol, ethyl acetate, mixture thereof. The ratio between the volume of crystallization solvent (in mL) and the mass of diol (in g) preferably ranges from 1 to 50, preferably from 2 to 30.

After crystallization, if performed, the method may further comprise a step a-ter) to wash the diol, for example with a solvent e.g. heptane.

The wash step allows reducing of the residual content of monoalcohols.

### Esterification Reaction

The esterification reaction can be performed with a homogeneous catalyst, a heterogeneous catalyst and/or enzymatic catalyst. It is also possible to perform the esterification reaction without a catalyst. For example, the catalyst can be a supported enzymatic catalyst.

The esterification reaction can be conducted using any method well known to skilled persons for the esterification of alcohols.

The catalyst for esterification can be chosen from among Lewis acid-based catalysts e.g. tin, titanate or boron trifluoride type.

As examples, the catalyst can be paratoluene sulfonic acid (p-TSA), methanesulfonic acid (MSA), sulfuric acid, boron trifluoride etherate (BF3·EtO), tin tetrachloride (FASCAT 4400), tin dichloride (FASCAT 2004), dibutyltin dichloride (FASCAT 4210), monobutyltin oxide (FASCAT 4100, TIB KAT 256), dibutyltin oxide (FASCAT 4201, TIB KAT 248), dioctyltin oxide (FASCAT 8201, TIB KAT 232), monobutyltin tris(2-ethylhexanoate) (FASCAT 4102), stannous oxalate (FASCAT 2001, TIB KAT 160), dibutyltin diacetate (FASCAT 4200, TIB KAT 233), dioctyltin diacetate (TIB KAT 223) and dioctyltin dicarboxylate (TIB KAT 318).

In one embodiment, the esterification step b) of the method of the invention is conducted at a temperature ranging from 50 to 200° C.

In one embodiment, the esterification reaction uses a diol of formula (3) and one or more saturated acids of formula (2), according to the following scheme:

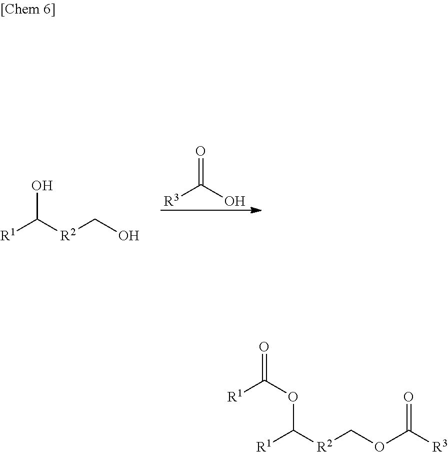

where R¹, R² and R³ have the same definition as in formulas (1) and (2) respectively, with the proviso that the groups R³ in the formed diester can be the same or different.

In particular, if two different saturated acids are used, the groups R³ in the formed diester can be the same or different.

In one embodiment, the reaction of the diol with the saturated acid in the presence of the catalyst is implemented under one or more of the following conditions:
the molar ratio of diol to saturated acid ranges from 1:2 to 1:10, preferably from 1:2 to 1:8, more preferably from 1:2 to 1:6;
the catalyst for the esterification reaction with the fatty acid(s) is used in a proportion ranging from 0.01 to 3.0 weight %, preferably 0.02 to 2.5 weight % relative to the total weight of the reaction medium.

Contrary to production methods of estolides or diesters having recourse to unsaturated compounds, as described in document US 2015/0094246, the method of the invention does not lead to a mixture of position isomers as a function of the position of the addition of the saturated fatty acid on the carbon-carbon double bond of the unsaturated compound. In the prior art, the saturated fatty acid can react on either of the carbon atoms of the carbon-carbon double bond of the unsaturated compound, which then leads to two position isomers. In addition, one portion of the unsaturated compounds can be isomerized under the reaction conditions of acid addition, which means that the carbon-carbon double bond may change position for some of the unsaturated compounds. Also, under the reaction conditions, this double bond may migrate along the alkyl chain, leading to a large number of isomers, differing by the position of functionalization, and to the possible formation of lactones.

Unlike these prior art methods, the method of the invention leads to a diester composition free of position isomers, insofar as the addition reaction of the saturated fatty acid selectively takes place solely at a position fixed by the hydroxyl function of the hydroxycarboxylic acid.

The diesters able to be obtained after the method can be represented by formula (4):

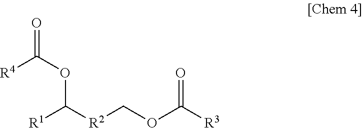

where:
R¹ is a linear or branched alkyl or alkenyl radical having 1 to 22 carbon atoms, preferably 3 to 18 carbon atoms, more preferably 5 to 12 carbon atoms,
R² is a divalent linear or branched alkyl or alkenyl radical having 1 to 22 carbon atoms, preferably 4 to 22 carbon atoms, more preferably 8 to 18 carbon atoms,
with the proviso that the total number of carbon atoms of R¹ and R² ranges from 9 to 23, preferably 9 to 20 carbon atoms, more preferably from 10 to 18 carbon atoms,
R³ and R⁴ are each independently a monovalent linear or branched alkyl radical having 1 to 19 carbon atoms, or a monovalent linear or branched alkenyl radical having 1 to 18 carbon atoms, preferably a linear or branched alkyl or alkenyl having 2 to 17 carbon atoms, advantageously a linear alkyl or alkenyl having 2 to 12 carbon atoms.

In one embodiment, the method of the invention allows the preparation of a diester of formula (4) where R³ et R⁴ are each independently a monovalent linear or branched alkyl radical having 1 to 19 carbon atoms, or a monovalent linear or branched alkenyl radical having 12 to 17 carbon atoms.

By «composition resulting from the method», consideration is given to the reagents, products and by-products of the reaction. The catalyst is not taken into consideration when designating the composition resulting from the method. In general therefore, the catalyst must be separated from the reaction medium to obtain the diester composition resulting from the method.

The method can be implemented continuously, semi-continuously or in batch mode.

The progression of the reaction can be monitored by gas phase chromatography with flame-ionization detection (GC-FID), by high performance liquid chromatography (HPLC) or by infrared according to methods known to skilled persons.

The diester composition resulting from the method advantageously has kinematic viscosity at 100° C. ranging from 1 to 50 mm²/s, preferably from 2 to 25 mm²/s, advantageously from 3 to 10 mm²/s, measured according to standard ASTM D445.

It is to be noted that the diester composition may optionally comprise from 0.1 to 30% by weight of non-reacted or intermediate reagents (monoesters of the diol or monoalcohols) optionally formed in situ, relative to the total weight of the diester composition, preferably from 0.2 to 10% by weight, more preferably 0.5 to 5% by weight.

The method of the invention, after the diester forming reaction, may optionally further comprise a separation step at which the non-reacted reagents of saturated fatty acid, hydroxycarboxylic acid and/or hydroxycarboxylic acid ester type, are removed from the composition de diesters. In the meaning of the present invention, the diesters are not reagents.

In one embodiment, the method of preparing at least one diester consists of the steps:
a) a conversion reaction of the acid function of at least one hydroxycarboxylic acid, or of the ester function of at least one ester of hydroxycarboxylic acid and alcohol, to an alcohol function to obtain at least one diol, said hydroxycarboxylic acid having 11 to 25 carbon atoms;
b) an esterification reaction of the diol by at least one saturated or unsaturated acid having 2 to 24 carbon atoms, to form at least one diester.

In one embodiment, the method of preparing at least one diester consists of the steps:
a) a conversion reaction of the acid function of at least one hydroxycarboxylic acid, or of the ester function of at least one ester of hydroxycarboxylic acid and alcohol, to an alcohol function to obtain at least one diol, said hydroxycarboxylic acid having 11 to 25 carbon atoms,
a-bis) a purification step of the composition of diols obtained after step a) to increase the content of diols in the diol composition obtained after step a), said purification step a-bis) preferably comprising at least one crystallization step of the diol and preferably being followed by one or more wash steps,
b) an esterification reaction of the diol by at least one saturated or unsaturated acid having 2 to 24 carbon atoms, to form at least one diester.

In one embodiment, the method of preparing at least one diester consists of the steps: 0) a preliminary step to purify a composition of hydroxycarboxylic acid or hydroxycarboxylic acid ester to increase the content of hydroxycarboxylic acid or hydroxycarboxylic acid ester in the composition of hydroxycarboxylic acid or hydroxycarboxylic acid ester, said preliminary purification step preferably comprising at least one crystallization step of the hydroxycarboxylic acid or ester thereof, and preferably being followed by one or more wash steps;
a) a conversion reaction of the acid function of at least one hydroxycarboxylic acid, or ester function of at least one ester of hydroxycarboxylic acid and alcohol, of the composition purified at step 0), to an alcohol function to obtain at least one diol, said hydroxycarboxylic acid having 11 to 25 carbon atoms;
b) an esterification reaction of the diol by at least one saturated or unsaturated acid having 2 to 24 carbon atoms, to form at least one diester.

### Diesters

A further subject of the present invention is a composition of diester(s) of formula (4) as such, and a composition of diester(s) able to be obtained with the method of the invention.

The composition of diester(s) of the invention comprises at least one diester meeting formula (4):

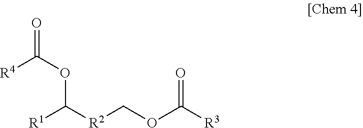

where:
R¹ is a linear or branched alkyl or alkenyl radical having 1 to 22 carbon atoms, preferably 3 to 18 carbon atoms, more preferably 5 to 12 carbon atoms;
R² is a divalent linear or branched alkyl or alkenyl radical having 1 to 22 carbon atoms, preferably 4 to 22 carbon atoms, more preferably 8 to 18 carbon atoms;
with the proviso that the total number of carbon atoms of R¹ and R² ranges from 9 to 23, preferably 9 to 20 carbon atoms, more preferably 10 to 18 carbon atoms,
R³ and R⁴ are each independently a monovalent linear or branched alkyl radical having 1 to 19 carbon atoms, or a monovalent linear or branched alkenyl radical having 1 to 18 carbon atoms, preferably a linear or branched alkyl or alkenyl having 2 to 17 carbon atoms, advantageously a linear alkyl or alkenyl having 2 to 12 carbon atoms.

The expression «said composition of diester(s) comprising, relative to the total weight of the composition of diester(s), at least 70% by weight of diesters of formula (4) only differing from each other by groups R³ and R⁴» is synonymous with the expression «said composition of diester(s) comprising, relative to the total weight of the composition of diester(s), at least 70% by weight of diesters of formula (4) in which the groups R¹ are the same and in which the groups R² are the same, being understood that R¹ may differ from R²». These two deux expressions mean that at least 70% by weight of the diesters(s) meet one same formula (4) in which solely the groups R³ and R⁴ may vary, the groups R¹ and R² remaining constant (for at least 70% by weight of the diesters).

In other words, the composition of diester(s) of the invention does not comprise or substantially does not comprise position isomers where the branching R³COO— (or R⁴COO—) at the chain R¹CHR²— could be positioned at different locations on said chain (as is the case with acid addition reactions on a carbon-carbon double bond).

In one embodiment of the diester composition of formula (4), if the diesters are unsaturated, then the diesters can be monounsaturated or polyunsaturated, the unsaturation(s) possibly being in one or more of the groups R¹, R², R³ and R⁴.

Preferably, if they are unsaturated, the radicals R³ and R⁴ are monounsaturated.

Preferably, in formula (4), only one of the radicals among R¹ and R² is monounsaturated, the other radical being saturated.

In one preferred embodiment, the composition of diester(s) of the invention comprises at least 75% by weight, preferably at least 80% by weight, more preferably at least 85% by weight of diesters of formula (4) in which the groups R¹ are the same and in which the groups R² are the same, being understood that R¹ may differ from R².

In one preferred embodiment:
R¹ is a linear alkyl having 5 to 8 carbon atoms, preferably a linear alkyl of 6 carbon atoms, and/or
R² is a linear alkyl having 8 to 12 carbon atoms, preferably a linear alkyl of 10 carbon atoms.

The diester composition of the invention advantageously has kinematic viscosity at 100° C. ranging from 1 to 50 mm²/s, preferably from 2 to 25 mm²/s, advantageously from 3 to 10 mm²/s, measured according to standard ASTM D445.

In one embodiment, the composition of diester(s) comprises from 70 to 99.8% by weight, preferably 75 to 99% by weight, more preferably 80 to 98% by weight of diester(s) of formula (4), relative to the total weight of the composition of diester(s).

In one particular embodiment, the composition of diester(s) of the invention comprises from 70 to 99.8% by weight, preferably 75 to 99% by weight, more preferably 80 to 98% by weight of diester(s) of formula (7), relative to the total weight of the composition of diester(s), the diesters meeting formula (7) are:

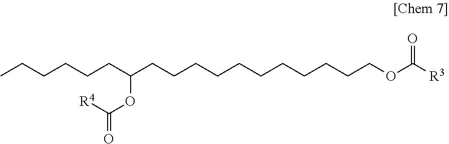

where:
R³ and R⁴ are each independently a monovalent linear or branched alkyl radical having 1 to 19 carbon atoms, or a monovalent linear or branched alkenyl radical having 1 to 18 carbon atoms, preferably a linear or branched alkyl having 2 to 17 carbon atoms, advantageously a linear or branched alkyl having 2 to 12 carbon atoms.

In one embodiment, the composition of diester(s) of the invention comprises at least one diester of formula (4), for example at least one diester of formula (7), where R³ and R⁴ are each independently a monovalent linear or branched alkyl radical having 1 to 19 carbon atoms, or a monovalent linear or branched alkenyl radical having 12 to 17 carbon atoms.

The composition of diester(s) of the invention is preferably obtained with a method of the invention. In particular, the composition of diester(s) of the invention typically does not comprise position isomers since it does not have recourse to unsaturated compounds such as unsaturated alcohols undergoing an addition reaction of a saturated acid on the carbon-carbon double bond.

### Uses

With the method of the invention, it is possible to obtain a diester composition having strong selectivity for a single diester. The composition of diester(s) of the invention can therefore be used as base oil in a lubricant composition.

The diester composition can be used in a lubricant composition as single base oil, but advantageously in combination with another base oil. By «another base oil», it is to be understood a base oil differing from the diesters.

The lubricant composition comprising the diester composition of the invention can be used to lubricate different parts of a vehicle, in particular the different parts of a vehicle engine or transmission, or the different parts of a marine engine or industrial machine engine e.g. for public works.

The diester composition able to be obtained can also be used as emollient in a cosmetic or pharmaceutical composition, alone or in combination with another fat. By «another fat», it is to be understood a fat differing from the diesters of the invention.

The cosmetic or pharmaceutical composition comprising the diester composition of the invention can be used for topical application, typically to the skin, nails, lips, hair, and scalp.

A further subject of the invention is the (non-therapeutic) cosmetic or pharmaceutical use of the diester composition of the invention as skincare product (serums, creams, balms, etc.), as hygiene product, as sunscreen/after-sun product, as make-up product, as make-up removal product, as fragranced product, as anti-perspirant product.

A further subject of the invention is a (non-therapeutic) cosmetic or pharmaceutical treatment method of the skin, nails, lips, hair or scalp, comprising at least one step of applying to the skin, nails, lips, hair or scalp a diester composition of the invention.

Finally, the invention also covers a cosmetic treatment method comprising at least one step of applying, preferably by spreading, the diester composition of the invention to the skin, nails, lips, hair or scalp.

### Lubricant Composition

A further subject of the invention is a lubricant composition comprising the composition of diester(s) of the invention and at least one additive and/or at least one other base oil.

Preferably, the composition of diester(s) used in the lubricant composition has one or more of the characteristics defined above in connection with the composition of diester(s). In one particular embodiment, the composition of diester(s) of the invention used in the lubricant composition comprises at least one saturated diester.

In one particular embodiment, the composition of diester(s) used in the lubricant composition of the invention meets formula (4) where R³ and R⁴ are each independently monovalent linear or branched alkyl radical having 1 to 19 carbon atoms, preferably a linear or branched alkyl having 2 to 17 carbon atoms, advantageously a linear alkyl having 2 to 12 carbon atoms.

These other base oils can be chosen from among base oils conventionally used in the field of lubricant oils, such as mineral, synthetic or natural oils, animal or vegetable oils, or mixtures thereof.

The other base oils of the lubricant compositions of the invention can in particular be oils of mineral or synthetic origin belonging to Groups I to V in the classes defined by the API classification (or equivalents thereof in the ATIEL classification), or mixture thereof, and given below in Table 1.

TABLE 1

| | Saturates content | Sulfur content | Viscosity |
|---|---|---|---|
| | (by weight) | (by weight) | index (VI) |
| Group I | <90% | >0.03% | 80 ≤ VI < 120 |
| Mineral oils | | | |
| Group II | ≥90% | ≤0.03% | 80 ≤ VI < 120 |
| Hydrocracked oils | | | |
| Group III | ≥90% | ≤0.03% | ≥120 |
| Hydrocracked or | | | |
| hydro-isomerized oils | | | |

| | |
|---|---|
| Group IV | Polyalphaolefins (PAO) |
| Group V | Esters and other bases not included in Groups I to IV |

The other mineral base oils include all types of base oils obtained by atmospheric and vacuum distillation of crude oil, followed by refining operations such as solvent extraction, deasphalting, solvent dewaxing, hydrotreatment, hydrocracking, hydroisomerization and hydrofinishing.

Mixtures of synthetic and mineral oils possibly being biosourced can also be used.

The other base oils of lubricant compositions of the invention can also be chosen from among synthetic oils such as some esters of carboxylic acids and alcohols, polyalphaolefins (PAOs), and polyalkylene glycols (PAG) obtained by polymerization or copolymerization of alkylene oxides having 2 to 8 carbon atoms, in particular 2 to 4 carbon atoms.

The PAOs used as other base oils are obtained for example from monomers having 4 to 32 carbon atoms, for example from octene or decene. The weight average molecular weight of the PAO can vary quite widely. Preferably, the weight average molecular weight of the PAO is lower than 600 Da. The weight average molecular weight of the PAO can range from 100 to 600 Da, from 150 to 600 Da, or from 200 to 600 Da. Advantageously, for low viscosity applications, the choice is made between PAO 2 and/or PAO 4.

Advantageously, the other base oil(s) of the lubricant composition of the invention are chosen from among polyalphaolefins (PAO), polyalkylene glycols (PAG) and the esters of carboxylic acids and alcohols.

In one alternative embodiment, the other base oil(s) of the lubricant composition of the invention can be chosen from among the base oils in Group II or III.

Skilled persons are able to adjust the content of base oil to be used in a lubricant composition.

In one embodiment, the lubricant composition of the invention comprises:
from 5 to 95 weight %, preferably 10 to 70 weight %, advantageously 15 to 50 weight % of the composition of diester(s) of the invention, and
from 5 to 95 weight %, preferably 30 to 90 weight %, advantageously 50 to 85 weight % of one or more other base oils,
relative to the total weight of the lubricant composition of the invention.

In one embodiment, the additive(s) of the lubricant composition are chosen from among friction modifiers, detergents, antiwear additives, extreme pressure additives, dispersants, antioxidants, pour point depressants, defoaming agents, metal passivators and mixtures thereof. These additives are well known to skilled persons in the field of lubrication of mechanical parts.

These additives can be added alone and/or in the form of a mixture such as vehicle engine lubricant formulations already commercially available meeting the performance requirements defined by ACEA (European Automobile Manufacturers' Association) and/or API (American Petroleum Institute), well known to skilled persons.

A lubricant composition of the invention may comprise at least one friction modifying additive. The friction modifier can be chosen from among a compound contributing metal elements and an ash-free compound. Among metal-contributing compounds mention can be made of complexes of transition metals such as Mo, Sb, Sn, Fe, Cu, Zn having ligands that are possibly hydrocarbon compounds comprising oxygen, nitrogen, sulfur, or phosphorus atoms. Ash-free friction modifiers are generally of organic origin can be chosen from among the monoesters of fatty acids and polyols, alkoxylated amines, alkoxylated fatty amines, fatty epoxides, borate fatty epoxides; fatty amines or fatty acid glycerol esters. In the invention, the fatty compounds comprise at least one hydrocarbon group having 10 to 24 carbon atoms.

A lubricant composition of the invention may comprise from 0.01 to 2% by weight or 0.01 to 5% by weight, preferably 0.1 to 1.5% by weight or 0.1 to 2% by weight of friction modifying additive relative to the total weight of the lubricant composition.

A lubricant composition used in the invention may comprise at least one antioxidant additive.

An antioxidant additive generally allows delayed degradation of the composition when in service. This degradation can particularly translate as the formation of deposits, the presence of sludge or an increase in the viscosity of the composition.

Antioxidant additives mainly act as radical inhibitors or hydroperoxide decomposers. Among antioxidant additives routinely used, mention can be made of antioxidant additives of phenolic type, antioxidant additives of amine type, sulfur-phosphorus antioxidant additives. Some of these antioxidant additives, for example those comprising sulfur and phosphorus can generate ash. Phenolic antioxidant additives can be ash-free or can be in the form of neutral or basic metal salts. The antioxidant additives can be chosen in particular from among sterically hindered phenols, sterically hindered phenol esters, and sterically hindered phenols comprising a thioether bridge, diphenylamines, diphenylamines substituted by at least one C₁-C₁₂ alkyl group, N,N′-dialkyl-aryl-diamines and mixtures thereof.

Preferably, in the invention, the sterically hindered phenols are chosen from among compounds comprising a phenol group in which at least one vicinal carbon of the carbon atom carrying the alcohol function is substituted by at least one C₁-C₁₀, alkyl group, preferably a C₁-C₆ alkyl group, preferably a C₄ alkyl group, preferably a tert-butyl group.

Amine compounds are another class of antioxidant additives which can be used, optionally in combination with phenolic antioxidant additives. Examples of amine compounds are aromatic amines e.g. aromatic amines having the formula NQ1Q2Q3 where Q1 is an aliphatic group or aromatic group, optionally substituted, Q2 is an optionally substituted aromatic group, Q3 is a hydrogen atom, alkyl group, aryl group or group of formula Q4S(O)ZQ5 where Q4 is an alkylene group or alkenylene group, Q5 is an alkyl group, alkenyl group or aryl group and z is 0, 1 or 2.

Sulfurized alkyl phenols or the alkali or alkaline-earth metal salts thereof can also be used as antioxidant additives

Another class of antioxidant additives includes copper-containing compounds, for example copper thio- or dithio-phosphates, the salts of copper and carboxylic acids, dithiocarbamates, sulfonates, phenates, copper acetylacetonates. The salts of copper I and II, the salts of succinic acid or anhydride can also be used.

A lubricant composition of the invention may contain all types of antioxidant additives known to skilled persons.

Advantageously, a lubricant composition of the invention comprises at least one ash-free antioxidant additive.

A lubricant composition of the invention may comprise from 0.5 to 2% by weight of at least one antioxidant additive, relative to the total weight of the composition.

A lubricant composition of the invention may also comprise at least one detergent additive.

Detergent additives generally allow a decrease in the formation of deposits on the surface of metal parts by dissolving secondary oxidation and combustion products.

The detergent additives able to be used in the lubricant composition of the invention are generally known to skilled persons. The detergent additives can be anionic compounds comprising a long lipophilic hydrocarbon chain and a hydrophilic head. The associated cation can be a metal cation of an alkali or alkaline-earth metal.

The detergent additives are preferably chosen from among the alkali metal or alkaline-earth metal salts of carboxylic acids, sulfonates, salicylates, naphthenates, and phenate salts. The alkali and alkaline-earth metals are preferably calcium, magnesium, sodium, or barium.

These metal salts generally comprise the metal in stoichiometric amount or else in excess, hence in an amount greater than the stoichiometric amount. In this case, they are overbased detergents; the excess metal imparting the overbased nature to the detergent additive is then generally in the form of an oil-insoluble metal salt e.g. a carbonate, hydroxide, an oxalate, acetate, a glutamate, preferably a carbonate.

A lubricant composition of the invention may for example comprise from 2 to 4% by weight of detergent additive, relative to the total weight of the composition.

In addition, a lubricant composition of the invention may comprise at least one dispersant, differing from the compounds of succinimide type defined in the invention.

The dispersant can be chosen from among Mannich bases, succinimides, e.g. of polyisobutylene succinimide type.

A lubricant composition used in the invention may comprise for example from 0.2 to 10 weight % of dispersant(s) differing from the compounds of succinimide type defined in the invention, relative to the total weight of the composition.

A lubricant composition of the invention may also comprise at least one antiwear and/or extreme pressure agent.

There exists a wide variety of antiwear additives. Preferably, for the lubricating composition of the invention, the antiwear additives are chosen from among organic phosphates. These have the advantage of not forming ash and of being heat stable. For example, mention can be made of sulfur-phosphorus additives such as metal alkylthiophosphates in particular zinc alkylthiophosphates, and more specifically zinc dialkyldithiophosphates or ZnDTP. The preferred compounds have the formula Zn((SP(S)(OQ6)(OQ7))2, where Q6 and Q7, the same or different, are each independently an alkyl group, preferably an alkyl group having 1 to 18 carbon atoms.

Amine phosphates are also antiwear and extreme pressure additives able to be used in a composition of the invention. However, the phosphorus contributed by these additives can act as poison for the catalytic systems of automotive vehicles since these additives generate ash. These effects can be minimised by partially substituting the amine phosphates by additives not contributing phosphorus such as polysulfides for example, in particular sulphur-containing olefins.

A lubricant composition of the invention may comprise from 0.01 to 15% by weight, preferably 0.1 to 10% by weight, more preferably from 1 to 5% by weight of antiwear agent(s) relative to the total weight of the composition.

A lubricant composition of the invention may also comprise at least one defoaming agent.

The defoaming agent can be chosen from among polyacrylates, polysiloxanes or the hybrids thereof.

A lubricant composition of the invention may comprise from 0.01 to 2 weight %, or from 0.01 to 5 weight %, preferably from 0.1 to 1.5 weight % or from 0.1 to 2 weight % of defoaming agent, relative to the total weight of the composition.

A lubricant composition suitable for the invention may also comprise at least one pour point depressant (PPD).

By slowing the formation of paraffin crystals, pour point depressant agents generally improve the cold start behaviour of the composition. As examples of pour point depressants, mention can be made of alkyl polymethacrylates, polyacrylates, polyarylamides, polyalkylphenols, polyalkylnaphthalenes, alkylated polystyrenes.

The lubricant composition of the invention may comprise:
from 5 to 94.9 weight %, preferably 10 to 70 weight %, advantageously 15 to 50 weight % of the composition of diester(s) of the invention, and
from 5 to 94.9 weight %, preferably from 30 to 90 weight %, advantageously 50 to 85 weight % of one or more other base oils,
from 0.1 to 15 weight %, preferably 0.5 to 10 weight %, advantageously from 1 to 5 weight % of one or more additives chosen from among friction modifiers, viscosity index modifiers, detergents, dispersants, antiwear and/or extreme pressure additives, antioxidants, pour point depressants, defoaming agents and mixtures thereof,
relative to the total weight of the lubricant composition of the invention.

The lubricant composition of the invention may comprise:
from 5 to 94.9 weight %, preferably 10 to 70 weight %, advantageously from 15 to 50 weight % of the composition of diester(s) of the invention comprising at least one diester of formula (4) where R³ and R⁴ are each independently monovalent linear or branched alkyl radical having 1 to 19 carbon atoms, preferably a linear or branched alkyl having 2 to 17 carbon atoms, advantageously a linear alkyl having 2 to 12 carbon atoms, and
from 5 to 94.9 weight %, preferably from 30 to 90 weight %, advantageously 50 to 85 weight % of one or more other base oils,
from 0.1 to 15 weight %, preferably from 0.5 to 10 weight %, advantageously 1 to 5 weight % of one or more additives chosen from among friction modifiers, viscosity index modifiers, detergents, dispersants, antiwear and/or extreme pressure additives, antioxidants, pour point depressants, defoaming agents and mixtures thereof,
relative to the total weight of the lubricant composition of the invention.

The lubricant composition of the invention can be obtained by mixing the constituents of the lubricant composition. The present invention also concerns a method of preparing a lubricant composition comprising the steps of:
preparing a composition of diester(s) following the above-described method, and
mixing at least one other base oil and/or at least one additive with the composition of diester(s).

Preferably, the method of preparing a lubricant composition of the invention does not comprise an intermediate step to separate the products formed at the preparation step of the composition of diester(s), before the mixing step. Preferably, the method of preparing a lubricant composition of the invention does not comprise hydrogenation step, in particular hydrogenation of the composition of diester(s) resulting from the preparation step of the composition of diester(s).

The other base oil(s) and the additive(s) used in the method of preparing the lubricant composition can have one or more of the characteristics previously described in connection with the lubricant composition of the invention.

The lubricant composition obtained with this preparation method may have one or more of the characteristics previously described for the lubricant composition of the invention.

### Cosmetic or Pharmaceutical Composition

A further subject of the invention is a cosmetic or pharmaceutical composition comprising (i) the composition of diester(s) of the invention and (ii) at least one fat and/or (iii) at least one cosmetic additive.

Preferably, the composition of diester(s) used in the cosmetic or pharmaceutical composition has one or more of the characteristics defined above for the composition of diester(s). In one particular embodiment, the composition of diester(s) of the invention used in the cosmetic or pharmaceutical composition comprises at least one unsaturated diester.

In one particular embodiment, the composition of diester(s) used in the cosmetic or pharmaceutical composition comprises at least one diester of formula (4) where R³ and R⁴ are each independently a monovalent linear or branched alkenyl radical having 1 to 18 carbon atoms, preferably 2 to 17 carbon atoms, more preferably 12 to 17 carbon atoms.

The fat can be chosen from among hydrocarbon oils of biological or petrochemical origin, vegetable oils, vegetable butters, ethers and fatty alcohols, oily esters (differing from the diesters of the invention), alkanes and silicone oils.

The hydrocarbon oils are fats derived from petrochemical processes. For example mention can be made of mineral oils, isoparaffins, waxes, paraffins, polyisobutenes or polydecenes.

Examples of vegetable oils are in particular wheat germ, sunflower seed, grapeseed, sesame, corn, apricot, castor, shea, avocado, olive, soybean oils; sweet almond, palm, rapeseed, hazelnut, macadamia, jojoba, alfalfa, poppy, pumpkin, sesame, gourd, rapeseed, blackcurrant, evening primrose, millet, barley, quinoa, rye, safflower, candlenut, passionflower, rosehip, or camellia oils. Vegetable butters are fats having the same properties as vegetable oils. The difference between them is the fact that butters are in solid form at ambient temperature. Therefore, unlike oils, the raw material from which butter is extracted (pulp, seeds or kernels) is heated after grinding to extract the fat. Like vegetable oils, butters can be refined to ensure better preservation, odour neutralization, improved colour and consistency. High in antioxidants and nourishing, the cosmetic properties of vegetable butters improve skin elasticity, protect against harsh external conditions leaving a protective film on the epidermis and thereby reduce dehydration, repair and soothe by regenerating the natural hydrolipid film of the skin. Examples of vegetable butters are in particular shea butter, cocoa butter, mango butter, shorea butter or olive butter.

Ethers and fatty alcohols are long-chain waxy fat substances having remarkable properties in particular film-forming, emollient, hydrating, smoothing and protective properties. The act as hydrating oils and as emulsifiers. Examples of fatty alcohols or ethers are: cetyl alcohol, stearyl alcohol, myristyl alcohol, lauryl alcohol, behenyl alcohol, cetearyl alcohol, dicaprylyl ethers, stearyl ethers or octyldodecanol (identified by their INCI name).

Oily esters or esterified oils (differing from the diesters of the invention) are the product of a reaction between fatty acids (longer chain acids such as stearic acid, oleic acid, palmitic acid) and alcohols (fatty alcohols or polyols such as glycerol). These oils may contain substances derived from petrochemical processes as is the case for isopropyl palmitate. Examples of oily esters are caprylic capric triglyceride, coco caprylate caprate, oleyl erucate, oleyl linoleate, decyl oleate or PPG-3 benzyl ether myristate (identified by their INCI name).

By silicone oils or polysiloxanes, it is meant an oil comprising at least one silicon atom, and in particular at least one group Si—O. As silicone oil, particular mention can be made of phenylpropyldimethylsiloxysilicate, dimethicones or cyclopentasiloxane (identified by their INCI name).

The additive, differing from the fat and the composition of diester(s), can be chosen from among any adjuvant or additive usually used in the fields under consideration and in particular in the cosmetic, dermatological, or pharmaceutical sectors. Evidently, skilled persons will take care to choose any additives to the composition of the invention such that the advantageous properties intrinsically attached to the emollient composition conforming to the invention are not or are not substantially deteriorated by the envisaged addition. Among conventional additives able to be included (depending the water-soluble or liposoluble nature of these additives), particular mention can be made foaming surfactants that can be anionic (such as sodium lauryl ether sulfate, sodium alkyl phosphate, sodium trideceth sulfate), amphoteric (such as alkyl betaine, disodium cocoamphodiacetate) or non-ionic having a HLB higher than 10 (such as POE/PPG/POE, Alkylpolyglucoside, polyglyceryl-3hydroxylauryl ether); preserving agents; sequestrants (EDTA); antioxidants; fragrances; colouring materials such as soluble colourants, pigments and pearls; mattifying fillers, tensors, whiteners or peeling agents; cosmetic active substances having the effect of improving the cosmetic properties of the skin, hydrophilic or lipophilic; electrolytes; hydrophilic or lipophilic, anionic, non-ionic, cationic or amphoteric polymers; thickeners, gelling agents or dispersants; slimming agents such as caffein; optical brighteners; anti-seborrheic agents; and mixture thereof. The amounts of these different cosmetic additives are those conventionally used in the field under consideration, for example the cosmetic composition has an overall content ranging from 0.01 to 20 weight % of additives relative to the total weight of the composition.

If the cosmetic, dermatological, pharmaceutical composition of the invention is a dermatological or pharmaceutical composition, said composition may comprise one or more therapeutic active ingredients. As active ingredients able to be used in the dermatological or pharmaceutical composition of the invention, mention can be made for example of sun filters; water-soluble or liposoluble vitamins such as vitamin A (retinol), vitamin E (tocopherol), vitamin C (ascorbic acid), vitamin B5 (panthenol), vitamin B3 (niacinamide), the derivatives of these vitamins (esters in particular) and mixtures thereof; antiseptics; antibacterials such as 2,4,4′-trichloro-2′-hydroxy diphenyl ether (or triclosan), 3,4,4′-trichlorocarbanilide (or triclocarban); antimicrobials such as benzoyl peroxide, niacin (vit. PP); and mixtures thereof.

This cosmetic or pharmaceutical composition comprises a physiologically acceptable medium i.e. not having any harmful adverse effects and in particular not producing redness, burning, tightness or stinging unacceptable for a user.

In one embodiment the cosmetic, dermatological, or pharmaceutical composition has a content of composition of diester(s) of the invention ranging from 0.5 to 80%, preferably from 1 to 50% and advantageously from 5 to 30 weight % relative to the total weight of the cosmetic or pharmaceutical composition.

In one embodiment of the invention, the cosmetic or pharmaceutical composition, relative to the total weight of the cosmetic or pharmaceutical composition, comprises:
0.5 to 80 weight %, preferably 1 to 50 weight % and advantageously from 5 to 30 weight % of the composition of diester(s) of the invention,
0 to 90 weight %, preferably 5 to 80 weight % and advantageously 10 to 70 weight %, preferably 20 to 60 weight % and advantageously 30 to 50 weight % of fats,
0 to 20 weight % of additives,
0 to 20 weight % of therapeutic active ingredients.
with the proviso that the composition comprises at least one additive or at least one fat.

In one embodiment of the invention, the cosmetic or pharmaceutical composition, relative to the total weight of the cosmetic or pharmaceutical composition, comprises:
0.5 to 80 weight %, preferably 1 to 50 weight % and advantageously 5 to 30 weight % of the composition of diester(s) of the invention;
0 to 90 weight %, preferably 5 to 80 weight % and advantageously 10 to 70 weight %, preferably 20 to 60 weight % and advantageously 30 to 50 weight % of fat chosen from among hydrocarbon oils of biological or petrochemical origin, vegetable oils, vegetable butters, ethers and fatty alcohols, oily esters (differing from the diesters), alkanes and silicone oils;
0 to 20 weight % of additives chosen from among anionic, amphoteric or non-ionic foaming surfactants having a HLB higher than 10; preserving agents; sequestrants; antioxidants; fragrances; colouring materials; mattifying fillers, tensors, whiteners or peeling agents; hydrophilic or lipophilic cosmetic active substances having the effect of improving the cosmetic properties of the skin; electrolytes; hydrophilic or lipophilic, anionic, non-ionic, cationic or amphoteric polymers, thickeners, gelling agents or dispersants; slimming agents; optical brighteners; anti-seborrheic agents; and mixture thereof;
optionally 0 to 20 weight % of therapeutic active ingredients.
with the proviso that the composition comprises at least one additive or at least one fat.

The cosmetic or pharmaceutical composition of the invention can therefore be an anhydrous composition, an emulsion such as a water-in-oil emulsion (w/o), oil-in-water emulsion (o/w) or a multiple emulsion (in particular w/o/w or o/w/o), a nano-emulsion or a dispersion.

The cosmetic or pharmaceutical composition of the invention can be in the form of a cream of varying softness or a vaporizable emulsion, for example it can form a make-up composition or skin or lip cleanser, an after-sun composition, a skin massaging composition, a balm shower gel composition, an anti-perspirant composition, mask composition, reparative balm composition, scrub and/or exfoliating composition both for the face and hands (when containing exfoliating particles), a make-up composition, shaving composition, after-shave balm composition, fragrant composition, a composition for wipes or a vaporizable composition.

The cosmetic or pharmaceutical composition of the invention can also form a sunscreen composition when including at least one sun filter.

The cosmetic or pharmaceutical composition of the invention is a cosmetic composition when it solely imparts a cosmetic effect. Typically, the cosmetic composition of the invention is free of therapeutic active ingredients.

On the contrary, the cosmetic or pharmaceutical composition of the invention is a dermatological or pharmaceutical composition if it imparts a therapeutic effect. Typically, the dermatological or pharmaceutical composition of the invention comprises at least one therapeutic active ingredient chosen for example from among sun filters; antiseptics; antibacterials such as 2,4,4′-trichloro-2′-hydroxy diphenyl ether (or triclosan), 3,4,4′-trichlorocarbanilide (or triclocarban); antimicrobials such as benzoyl peroxide, niacin (vit. PP); and mixtures thereof.

### EXAMPLES

In the remainder of the present description, examples are given to illustrate the invention, and are not in any manner intended to limit the scope thereof.

### Example 1: Method of Preparing a Diester

Step a): Reduction of 12-hydroxystearic acid (12-HSA) to 1,12-octadecanediol

A solution is prepared of 60 g of 12-HSA in V2=250 ml of dry THF.

A three-neck flask is charged with a commercial solution of lithium aluminium hydride (LAH) followed by V1=250 mL of dry THF.

The mixture is cooled to −5° C.

Over a time of 30 min, the solution 12-HSA is added via a dropping funnel, without exceeding +10° C.

There is setting of the reaction medium. 200 mL de THF are added and the medium left under agitation overnight at ambient temperature until complete conversion of the starting product. Thin layer chromatography (SiO₂/aluminium; heptane 70/AcOEt 30/PMA) is used to indicate the end of the reaction.

Treatment: The medium is cooled to +10° C. Hydrolysis is carried out by dropwise addition of 250 ml (3.3 vol.) of potable water. The mixture is acidified to pH=2 with the addition of 180 mL (3 vol.) of 30% sulfuric acid. After decanting and separation of the two phases, the aqueous phase is extracted with 3×150 mL MTBE. The organic phases are grouped together and washed with 250 ml of potable water, 250 ml of saturated NaHCO₃ solution and 250 mL of brine. The organic phase is concentrated to dryness in a rotary evaporator at 45° C.

1,12-octadecanediol is obtained in the form of a white solid.

Step a-bis): Crystallization of 1,12-octadecanediol

The crude 1,12-octadecanediol is recrystallized by refluxing 5 to 20 g de diol in suspension in 30 to 200 mL of heptane. The mixture remains turbid and heptane is gradually added in portions of 10 mL leaving under reflux for 10 min between each addition. The mixture is left under slow agitation at ambient temperature for 2 hours. The diol starts to crystallize at around 50-55° C.

The precipitate is isolated by filtration on a N^{o} 4 sintered filter plate.

A white solid is obtained, dried for 2 h via nitrogen evaporation.

Crystallization allows the obtaining of 1,12-octadecanediol purity of at least 80%, even at least 90%.

Step a-ter): Washing

The solid obtained after step a-bis), mostly composed of 1,12-octadecanediol, is filtered through a temperature-controlled filter and the cake obtained is washed once with 6 volumes of cold heptane.

With this protocol, it is possible to obtain a residual content of fatty alcohols of less than 0.1 weight %.

Step b): Esterification of 1,12-octadecanediol to Diester

The 1,12-octadecanediol diol, optionally purified as described at step a-bis), is esterified by butyric acid in the presence of a catalyst (PTSA) in toluene with a diol/saturated acid weight ratio of 1:4.

### Example 2: Properties of the Diester

The diester obtained either without purification or with purification (crystallization at step a-bis) followed by washing at step a-ter)) was tested for the physiochemical properties thereof. The results are given in Table 2 and show that the diesters obtained with the method of the invention have properties making them suitable for lubricant applications.

TABLE 2

| | Diester | Mini pour | KV100 | Viscosity | Noack by |
|---|---|---|---|---|---|
| | purity | point (° C.) | (cSt) | index | ATG (%) |
| | (% | ASTM | ASTM | ASTM | ASTM |
| Purification | GC) | D7346 | D7042 | D2270 | D6375 |
| None | 83.2 | −6 | 3.424 | 150 | 12.7 |
| Crystallization | 98 | −24 | 3.586 | 150 | 10.1 |

### Example 3: Preparation and Evaluation of Other Diesters

Diesters were prepared with the same method as in Example 1 with implementation of the crystallization step a-bis), where at step b), butyric acid was replaced by another acid:
Ex. 3A=acetic acid/2-ethylhexanoic acid in a molar ratio of 1:3;
Ex. 3B=oleic acid.

The physicochemical properties of the diesters were measured and are given in Table 3.

TABLE 3

| | KV40 | KV100 | Viscosity | Mini pour | Noack by |
|---|---|---|---|---|---|
| | (cSt) | (cSt) | index | point (° C.) | ATG (%) |
| | ASTM | ASTM | ASTM | ASTM | ASTM |
| | D7042 | D7042 | D2270 | D7346 | D6375 |
| Ex. 3A | 14.74 | 3.68 | 141 | −54 | 11.2 |
| Ex. 3B | 56.4 | 11.11 | 194 | −9 | 0.31 |

The results in Table 3 show that the diesters of the invention display good physicochemical properties, in particular a good viscosity index allowing use thereof as lubricant base oil or as emollient in cosmetics. The diester in Ex. 3B prepared from a long chain unsaturated acid (e.g. more than 11 carbon atoms) can be given particular use as emollient in cosmetic compositions.

1.-15. (canceled)

16. A method for preparing at least one diester, said method comprising:
a) a transformation reaction of the acid function of at least one hydroxycarboxylic acid, or of the ester function of at least one ester of hydroxycarboxylic acid and alcohol, into an alcohol function to obtain at least one diol, said hydroxycarboxylic acid having 11 to 25 carbon atoms,
b) an esterification reaction of the diol via at least one saturated or unsaturated acid having 2 to 24 carbon atoms, to form at least one diester,
the diester meeting formula (4): 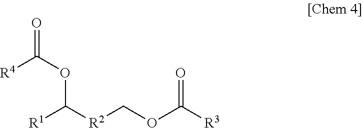
where:
R¹ is a linear or branched alkyl radical having 1 to 22 carbon atoms, or a linear or branched alkenyl radical having 2 to 22 carbon atoms,
R² is a divalent linear or branched alkyl or alkenyl radical having 4 to 22 carbon atoms,
with the proviso that the total number of carbon atoms of R¹ and R² ranges from 9 to 23,
R³ and R⁴ each independently are a monovalent linear or branched alkyl radical having 1 to 19 carbon atoms, or a monovalent linear or branched alkenyl radical having 1 to 18 carbon atoms.

17. The method according to claim 16, further comprising after step a) and before step b), a step a-bis) to purify the diol composition obtained after step a) to increase the diol content of the diol composition obtained after step a).

18. The method according to claim 16, further comprising before step a) a preliminary purification step of the hydroxycarboxylic acid composition or hydroxycarboxylic acid ester composition to increase the content of hydroxycarboxylic acid or hydroxycarboxylic acid ester in the composition of hydroxycarboxylic acid or hydroxycarboxylic acid ester used at subsequent step a).

19. The method according to claim 16, wherein the hydroxycarboxylic acid meets formula (1) and/or the saturated or unsaturated acid meets formula (2): 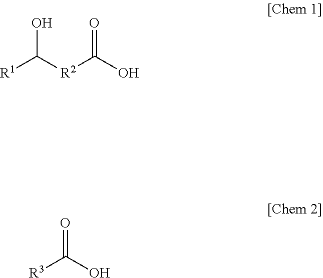
where:
R¹ is a linear or branched alkyl or alkenyl radical having 1 to 22 carbon atoms,
R² is a divalent linear or branched alkyl or alkenyl radical having 4 to 22 carbon atoms,
with the proviso that the total number of carbon atoms of R¹ and R² ranges from 9 to 23,
R³ is a monovalent linear or branched alkyl radical having 1 to 19 carbon atoms.

20. The method according to claim 16, wherein the diol meets formula (3) and the diester meets formula (4): 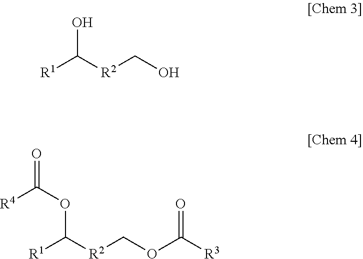
where:
R¹ is an alkyl or alkenyl radical having 3 to 18 carbon atoms,
R² is a divalent alkyl or alkenyl radical having 8 to 18 carbon atoms,
with the proviso that the total number of carbon atoms of R¹ and R² ranges from 9 to 20 carbon atoms,
R³ and R⁴ are each independently a monovalent linear or branched alkyl or alkenyl radical having 2 to 17 carbon atoms.

21. The method according to claim 16, wherein a mixture of saturated or unsaturated acid is used at step b).

22. A composition of diester(s) comprising at least one diester of formula (4): 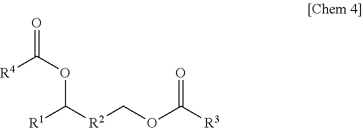
where:
R¹ is a linear or branched alkyl radical having 1 to 22 carbon atoms, or a linear or branched alkenyl radical having 2 to 22 carbon atoms;
R² is a divalent linear or branched alkyl or alkenyl radical having 4 to 22 carbon atoms,
with the proviso that the total number of carbon atoms of R¹ and R² ranges from 9 to 23;
R³ and R⁴ are each independently a monovalent linear or branched alkyl radical having 1 to 19 carbon atoms, or a monovalent linear or branched alkenyl radical having 1 to 18 carbon atoms;
said composition of diester(s), relative to the total weight of the composition of diester(s), comprising at least 70 weight % of diesters of formula (4) which only differ from each other by the groups R³ and R⁴.

23. The composition of diester(s) according to claim 22, obtained with the method for preparing at least one diester, said method comprising:
a) a transformation reaction of the acid function of at least one hydroxycarboxylic acid, or of the ester function of at least one ester of hydroxycarboxylic acid and alcohol, into an alcohol function to obtain at least one diol, said hydroxycarboxylic acid having 11 to 25 carbon atoms,
b) an esterification reaction of the diol via at least one saturated or unsaturated acid having 2 to 24 carbon atoms, to form at least one diester,
the diester meeting formula (4): 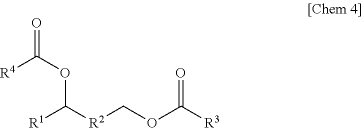
where:
R¹ is a linear or branched alkyl radical having 1 to 22 carbon atoms, or a linear or branched alkenyl radical having 2 to 22 carbon atoms,
R² is a divalent linear or branched alkyl or alkenyl radical having 4 to 22 carbon atoms,
with the proviso that the total number of carbon atoms of R¹ and R² ranges from 9 to 23,
R³ and R⁴ each independently are a monovalent linear or branched alkyl radical having 1 to 19 carbon atoms, or a monovalent linear or branched alkenyl radical having 1 to 18 carbon atoms.

24. The composition according to claim 22, comprising, relative to the total weight of the composition, at least 70 weight % of a single diester meeting formula (4).

25. The composition according to claim 22, wherein R³ and R⁴ each independently are a monovalent linear or branched alkyl radical having 1 to 19 carbon atoms, or a monovalent linear or branched alkenyl radical having 12 to 17 carbon atoms.

26. A method comprising a step of using the composition of diester(s) according to claim 22, as base oil in a lubricant composition, or as emollient in a cosmetic or pharmaceutical composition.

27. A lubricant composition comprising the composition of diester(s) according to claim 22, and:
(i) at least one base oil differing from the diesters, and/or
(ii) at least one additive differing from the diesters.

28. The lubricant composition according to claim 27, wherein the composition of diester(s) comprises at least one diester of formula (4) where R³ and R⁴ are each independently a monovalent linear or branched alkyl radical having 1 to 19 carbon atoms.

29. A cosmetic or pharmaceutical composition comprising (i) the composition of diester(s) according to claim 7 and (ii) at least one fat and/or (iii) at least one cosmetic additive.

30. The cosmetic or pharmaceutical composition according to claim 29, wherein the composition of diester(s) comprises at least one diester of formula (4) where R³ and R⁴ are each independently a monovalent linear or branched alkenyl radical having 1 to 18 carbon atoms.

31. The method according to claim 16, further comprising after step a) and before step b), a step a-bis) to purify the diol composition obtained after step a) to increase the diol content of the diol composition obtained after step a), said purification step a-bis) comprising at least one crystallization step of the diol.

32. The method according to claim 16, further comprising before step a) a preliminary purification step of the hydroxycarboxylic acid composition or hydroxycarboxylic acid ester composition to increase the content of hydroxycarboxylic acid or hydroxycarboxylic acid ester in the composition of hydroxycarboxylic acid or hydroxycarboxylic acid ester used at subsequent step a), said preliminary purification step comprising at least one crystallization step of the hydroxycarboxylic acid or hydroxycarboxylic acid ester.

33. The method according to claim 16, wherein the diol meets formula (3) and the diester meets formula (4): 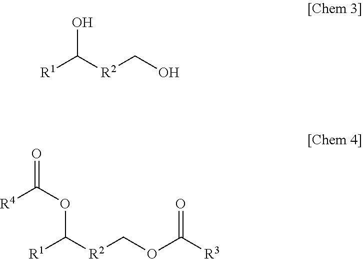
where:
R¹ is an alkyl or alkenyl radical having 5 to 12 carbon atoms,
R² is a divalent alkyl or alkenyl radical having 8 to 18 carbon atoms,
with the proviso that the total number of carbon atoms of R¹ and R² ranges from 10 to 18 carbon atoms,
R³ and R⁴ are each independently a monovalent linear alkyl or alkenyl having 2 to 12 carbon atoms.

34. The composition of diester(s) according to claim 22, comprising at least one diester of formula (4): 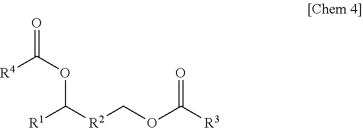
where:
R¹ is a linear or branched alkyl or alkenyl radical having 5 to 12 carbon atoms;
R² is a divalent linear or branched alkyl or alkenyl radical having 8 to 18 carbon atoms,
with the proviso that the total number of carbon atoms of R¹ and R² ranges from 10 to 18 carbon atoms;
R³ and R⁴ are each independently a monovalent linear alkyl or alkenyl having 2 to 12 carbon atoms;
said composition of diester(s), relative to the total weight of the composition of diester(s), comprising at least 70 weight % of diesters of formula (4) which only differ from each other by the groups R³ and R⁴.

35. The lubricant composition according to claim 27, wherein the composition of diester(s) comprises at least one diester of formula (4) where R³ and R⁴ are each independently a monovalent linear alkyl having 2 to 12 carbon atoms.
